(19) United States
(12) Patent Application Publication
(10) Pub. No.: US 2026/0335393 A1
(43) Pub. Date: Oct. 29, 2026

(54) Traditional Chinese medicine composition for treating immune vasculitis and formulation and use thereof

(71) Applicant: SHANGHAI TRADITIONAL CHINESE MEDICINE INTEGRATED HOSPITAL, Shanghai (CN)

(72) Inventors: Yemin Cao, Shanghai (CN); Cheng Zhao, Shanghai (CN); Ji Li, Shanghai (CN); Xia Feng, Shanghai (CN); Chenglin Jia, Shanghai (CN); Yuzhen Wang, Shanghai (CN)

(21) Appl. No.: 19/114,401

(22) PCT Filed: Sep. 16, 2023
(86) PCT No.: PCT/CN2023/119265

§ 371 Date: Mar. 21, 2025

(30) Foreign Application Priority Data
Sep. 23, 2022 (CN) 202211167437.5

(51) Int. Cl. A61K 36/64; A61K 33/06; A61K 35/10; A61K 35/32; A61K 36/258; A61K 36/41; A61K 36/484; A61K 36/634; A61K 36/65; A61K 36/708; A61K 36/748; A61K 36/81; A61P 37/06

(52) U.S. Cl. CPC A61K 36/64; A61K 33/06; A61K 35/10; A61K 35/32; A61K 36/258; A61K 36/41; A61K 36/484; A61K 36/634; A61K 36/65; A61K 36/708; A61K 36/748; A61K 36/81; A61P 37/06

(57) ABSTRACT

A traditional Chinese medicine composition for treating immune vasculitis, prepared from 15-40 parts of Radix rehmanniae, 10-20 parts of Radix paeoniae rubra, 10-40 parts of fructus gardeniae, 15-60 parts of gypsum, 15-40 parts of Sedum sarmentosum, 15-40 parts of Solanum lyratum, 15-40 parts of Oldenlandis diffusa, 15-40 parts of Cornu bubali, 10-20 parts of fructus forsythiae, 6-20 parts of fried Rheum officinale, 3-15 parts of liquorice, 0.6-2.4 parts of antelope horn, 3-12 parts of amber powder, and 2-8 parts of Panax notoginseng powder. The traditional Chinese medicine composition may be prepared into a decoction, a granule, a pluvise, a capsule, a tablet, or an oral liquid. The immuse vasculitics disease is nodular vasculitis, systemic lupus erythematosus vascultits, scleroderma vasculitis, or ACNA-related vasculitis.

### BACKGROUND OF THE PRESENT INVENTION

### Field of Invention

The present invention relates to the technical field of traditional Chinese medicine, and more particularly to a traditional Chinese medicine composition for treating immune vasculitis and formulation and use thereof.

### Description of Related Arts

Immune vasculitis refers to the inflammatory changes of the vascular wall and its surrounding tissues in histopathology. In severe cases, there will be thrombosis, even the destruction of the entire blood vessel, leading to hemorrhagic and/or ischemic lesions, and eventually tissue necrosis or infarction. Immunovascular inflammatory diseases are not uncommon in clinical practice. The results show that the annual incidence rate is in the range of 39.6/million to 59.8/million. Immune vasculitis diseases mainly include major Takayasu arteritis, giant cell arteritis, polyarteritis nodosa, ANCA (antineutrophil cytoplasmic antibodies)-associated vasculitis, granulomatosis with polyangiitis, Allergic Granulomatosis with Polyangiitis, Cryoglobulin vasculitis, IgA vasculitis, variant vasculitis, thromboangitis obliterans, vasculitis associated with systemic lupus erythematosus, vasculitis associated with Rheumatoid arthritis, vasculitis scleroderma, and vasculitis associated with sarcoidosis. At present, the treatment methods of different types of immune vasculitis are similar, or hormones or immunosuppressants are used. However, the treatment effect is still not ideal, and the side effects of hormones or immunosuppressants are large. Therefore, the research and development of therapeutic drugs for immune vasculitis is still the focus and difficulty of current research.

### SUMMARY OF THE PRESENT INVENTION

An object of the present invention is to provide a traditional Chinese medicine composition for treating immune vasculitis and formulation and use thereof.

Firstly, the present invention provides a traditional Chinese medicine composition for treating immune vasculitis, which includes following raw material components by weight:
15-40 parts of Radix rehmanniae, 10-20 parts of Radix paeoniae rubra, 10-40 parts of fructus gardeniae, 15-60 parts of gypsum, 15-40 parts of Sedum sarmentosum, 15-40 parts of Solanum lyratum, 15-40 parts of Oldenlandia diffusa, 15-40 parts of Cornu bubali, 10-20 parts of fructus forsythiae, 6-20 parts of fried Rheum officinale, 3-15 parts of liquorice, 0.6-2.4 parts of antelope horn, 3-12 parts of amber, and 2-8 parts of Panax notoginseng.

Preferably, the traditional Chinese medicine composition for treating immune vasculitis includes following raw material components by weight: 30 parts of Radix rehmanniae, 15 parts of Radix paeoniae rubra, 15 parts of fructus gardeniae, 30 parts of gypsum, 30 parts of Sedum sarmentosum, 30 parts of Solanum lyratum, 30 parts of Oldenlandia diffusa, 30 parts of Cornu bubali, 15 parts of fructus forsythiae, 12 parts of fried Rheum officinale, 9 parts of liquorice, 1.2 parts of antelope horn, 6 parts of amber, and 4 parts of Panax notoginseng.

Preferably, the traditional Chinese medicine composition for treating immune vasculitis includes following raw material components by weight: 20 parts of Radix rehmanniae, 10 parts of Radix paeoniae rubra, 20 parts of fructus gardeniae, 20 parts of gypsum, 20 parts of Sedum sarmentosum, 20 parts of Solanum lyratum, 20 parts of Oldenlandia diffusa, 20 parts of Cornu bubali, 10 parts of fructus forsythiae, 6 parts of fried Rheum officinale, 3 parts of liquorice, 0.6 parts of antelope horn, 3 parts of amber, and 2 parts of Panax notoginseng.

Preferably, the traditional Chinese medicine composition for treating immune vasculitis includes following raw material components by weight: 25 parts of Radix rehmanniae, 13 parts of Radix paeoniae rubra, 25 parts of fructus gardeniae, 25 parts of gypsum, 25 parts of Sedum sarmentosum, 25 parts of Solanum lyratum, 25 parts of Oldenlandia diffusa, 25 parts of Cornu bubali, 13 parts of fructus forsythiae, 9 parts of fried Rheum officinale, 6 parts of liquorice, 0.9 parts of antelope horn, 4.5 parts of amber, and 3 parts of Panax notoginseng.

Preferably, the traditional Chinese medicine composition for treating immune vasculitis includes following raw material components by weight: 35 parts of Radix rehmanniae, 17 parts of Radix paeoniae rubra, 35 parts of fructus gardeniae, 45 parts of gypsum, 35 parts of Sedum sarmentosum, 35 parts of Solanum lyratum, 35 parts of Oldenlandia diffusa, 45 parts of Cornu bubali, 17 parts of fructus forsythiae, 12 parts of fried Rheum officinale, 12 parts of liquorice, 1.8 parts of antelope horn, 9 parts of amber, and 6 parts of Panax notoginseng.

Preferably, the traditional Chinese medicine composition for treating immune vasculitis includes following raw material components by weight: 40 parts of Radix rehmanniae, 20 parts of Radix paeoniae rubra, 40 parts of fructus gardeniae, 60 parts of gypsum, 40 parts of Sedum sarmentosum, 40 parts of Solanum lyratum, 40 parts of Oldenlandia diffusa, 40 parts of Cornu bubali, 20 parts of fructus forsythiae, 20 parts of fried Rheum officinale, 15 parts of liquorice, 2.4 parts of antelope horn, 12 parts of amber, and 8 parts of Panax notoginseng.

Secondly, the present invention provides a Chinese herbal medicine compound preparation for treating immune vasculitis, which is a decoction, a granule, a pulvis, a capsule, a tablet, or an oral liquid with the above traditional Chinese medicine composition as an active component.

Preferably, the Chinese herbal medicine compound preparation is a decoction.

Also, the present invention provides a method for treating immune vasculitis disease comprising applying the above traditional Chinese medicine composition. The immune vasculitis disease is nodular vasculitis, aorto-arteritis, thromboangiitis obliterans, Rheumatoid vasculitis, systemic lupus erythematosus vasculitis, scleroderma vasculitis, or ACNA (antineutrophil cytoplasmic antibodies)-related vasculitis.

The details of various aspects of the present invention are described in detail in subsequent sections. The characteristics, purposes and advantages of the present invention will become more apparent by the following description and the claims.

### BRIEF DESCRIPTION OF THE DRAWINGS

FIG. 1 shows that the traditional Chinese medicine composition significantly improve the necrosis of lower limbs in rats, in which NC is the normal group, MOD is the model group, and LXHY is the Chinese medicine composition group.

FIG. 2 shows that the traditional Chinese medicine composition significantly increases the blood flow of lower limbs in rats with immune vasculitis, in which FIG. 2a is a typical picture, FIG. 2b shows the quantization result, NC is the normal group, MOD is the model group, LXHY is the Chinese medicine composition group, * represents P<0.05 compared with the normal group, # represents P<0.05 compared with the model group.

FIG. 3 shows that the traditional Chinese medicine composition has no obvious toxicity to the heart, liver and kidney, in which NC is the normal group, MOD is the model group, and LXHY is the Chinese medicine composition group.

FIG. 4 shows that the traditional Chinese medicine composition significantly reduces the infiltration of inflammatory cells, in which NC is the normal group, MOD is the model group, LXHY is the Chinese medicine composition group, and WXF is the literature group, * represents P<0.05 compared with the normal group, # represents P<0.05 compared with the model group, A represents P<0.05 compared with the literature group.

FIG. 5 shows that the traditional Chinese medicine composition significantly improves the lesion of elastic fibers, in which NC is the normal group, MOD is the model group, LXHY is the Chinese medicine composition group, and WXF is the literature group, * represents P<0.05 compared with the normal group, # represents P<0.05 compared with the model group, A represents P<0.05 compared with the literature group.

FIG. 6 shows that the effect of the traditional Chinese medicine composition on immune vasculitis is superior to that of any separated compound prescription, in which NC is the normal group, MOD is the model group, LXHY is the Chinese medicine composition group, CF1 represents the first separated compound prescription, CF2 represents the second separated compound prescription, CF3 represents the third separated compound prescription, CF4 represents the fourth separated compound prescription, CF5 represents the fifth separated compound prescription, CF6 represents the sixth separated compound prescription, CF7 represents the seventh separated compound prescription, CF8 represents the eighth separated compound prescription, CF9 represents the ninth separated compound prescription, CF10 represents the tenth separated compound prescription, CF11 represents the eleventh separated compound prescription, CF12 represents the twelfth separated compound prescription, CF13 represents the thirteenth, CF14 represents the fourteenth separated compound prescription, * represents P<0.05 compared with the normal group, # represents P<0.05 compared with the model group, A represents P<0.05 compared with LXHY.

### DETAILED DESCRIPTION OF THE PREFERRED EMBODIMENT

The Chinese medicinal materials used by the inventor in the process of preparing the medicine composition provided by the present invention are as follows: Radix rehmanniae (specifically fresh or dried roots of Rehmannia glutinosa Libosch.), Radix paeoniae rubra (specifically dried roots of Paeonia lactiflora Pall, or Paeonia veitchii Lynch), fructus gardeniae (specifically the dried and ripe fruit of Gardenia jasminoides Ellis), gypsum (specifically a sulfate mineral anhydrite family gypsum) mainly containing hydrous calcium sulfate CaSO₄·2H₂O, Sedum sarmentosum (specifically the whole plant of Sedum sarmentosum Bunge), Solanum lyratum (specifically the whole plant or root of Solanum lyratum Thunb), Oldenlandia diffusa (specifically the whole plant of Hedyotis diffusa Willd. [Oldenlandia diffusa (Willd.) Roxb.]), Cornu bubali (specifically the horn of Bubalus bubalis Linnaeus), fructus forsythiae (specifically the dried fruit of Forsythia suspensa (Thunb.) Vah) which is bitter and slightly cold, and belongs to the lung, heart, and small intestine, fried Rheum officinale (specifically the dried root and rhizome of Rheum palmatum L., R. tanguticum Maxim. Ex Balf or R. officinale Baill.), liquorice (specifically the dried root of Glycyrrhiza uralensis Fisch., glycyrrhiza inflata Bat. or Glycyrrhiza glabra L.), antelope horn (specifically the horn of Saiga tatarica Linnaeus) which is salty and cold, and belongs to the liver and heart meridian, amber which is a fossil-like substance formed by the resin of ancient pinaceae plants, such as maple and pine, buried underground for a long time), and Panax notoginseng (specifically the powder manufactured by the dried root of Panax notoginseng (Burk.) F. H. Chen).

Based on many years of clinical experience, the inventor believes that the pathogenesis of immune vasculitis is composed of “pathogenic factors, stagnation and damage”. In the acute stage, “pathogenic factors” is the main one, focusing on “eliminating pathogenic factors”, which are mainly manifested as pathogenic wind heat, pathogenic damp heat, heat toxin or heat in blood, so the treatment method is “cooling blood”. In the relief period, “pathogenic factors” have been removed, focusing on “removing the stagnation”. In the recovery period, the problems of “pathogenic factors and stagnation” have been solved, focusing on “recovery after damage”, so the treatment method is “nutrient recovery”. Furthermore, the traditional Chinese medicine composition provided by the present invention contains fourteen traditional Chinese medicines. Among them, Radix rehmanniae, Radix paeoniae rubra, fructus gardeniae, and gypsum are capable of clearing heat and resolving dampness, and are configured to eliminate pathogenic factors and play the important roles; Sedum sarmentosum, Solanum lyratum, Oldenlandia diffusa, Cornu bubali, fructus forsythiae, and antelope horn are capable of clearing away toxic materials for cooling blood; fried Rheum officinale, amber, and Panax notoginseng, acting as the auxiliary elements, are capable of resolving dampness and removing the stagnation; liquorice is capable of tonifying spleen and benefiting lung, so that the combination is achieved. All the above materials are combined with each other to achieve the desired effect.

The antelope horn and Panax notoginseng in the traditional Chinese medicine composition provided by the present invention are purchased from Shanghai Wanshicheng State Medicine Products Co., LTD, China, the other raw materials are granules prepared by China Resources Sanjiu Medical & Pharmaceutical Co., LTD. The traditional Chinese medicine composition provided by the present invention is able to be prepared according to the conventional method in the field. For example, the preparation method comprises steps of (S1) crushing antelope horn, amber and Panax notoginseng into powders; and (S2) obtaining a first mixed solution and a second mixed solution by adding 8 times and 6 times the amount of water to the remaining eleven raw materials respectively; decocting the first mixed solution and the second mixed solution twice respectively, wherein each time is one hour; obtaining a first filtrate and a second filtrate by filtering respectively; merging the first filtrate and the second filtrate, concentrating into an extract with a relative density in the range of 1.15-1.2 at 80-85 °C.

The traditional Chinese medicine composition provided by the present invention has the advantages of convenient use, exact curative effect, small side effect, low cost, long-term use, and good clinical curative effect in the treatment of different types of immune vasculitis. The traditional Chinese medicine composition has good clinical efficacy in the treatment of different types of immune vasculitis. In view of the high incidence of immune vasculitis, the huge physical and mental pain to patients, different treatment effects of the current hormones and immunosuppressants, and the limitations of easy recurrence and large side effects, the traditional Chinese medicine composition provided by the present invention has a good clinical application prospect.

The present invention is further explained in combination with specific embodiments as follows. It should be understood that these embodiments are used only to illustrate the present invention and not to limit the scope of the present invention. The experimental methods not specified in the following embodiments are usually in accordance with the conventional conditions or the conditions recommended by the manufacturer. Unless otherwise stated, all percentages, ratios, proportions, or portions are measured by weight.

Unless otherwise defined, all professional and scientific terms used in this specification have the same meaning as those familiar to those skilled in the art. In addition, any method or material similar or equal to the recorded content may be applied to the method provided by the present invention. The preferred implementation methods and materials described in this specification are only for demonstration.

The above features mentioned in the present invention, or the features mentioned in embodiments, may be arbitrarily combined. All features disclosed in the specification of the present invention may be used with any composition form, and each feature disclosed in the specification may be replaced by any alternative feature that provides the same, equal or similar purpose. Therefore, unless otherwise specified, the features revealed are only general examples of equal or similar features.

### First Embodiment

20 g of Radix rehmanniae, 10 g of Radix paeoniae rubra, 20 g of fructus gardeniae, 20 g of gypsum, 20 g of Sedum sarmentosum, 20 g of Solanum lyratum, 20 g of Oldenlandia diffusa, 20 g of Cornu bubali, 10 g of fructus forsythiae, 6 g of fried Rheum officinale, 3 g of liquorice, 0.6 g of antelope horn, 3 g of amber, and 2 g of Panax notoginseng are taken.

A preparation method comprises steps of:
(S1) crushing the above weight of antelope horn, amber and Panax notoginseng into powders; and
(S2) obtaining a first mixed solution and a second mixed solution by adding 8 times and 6 times the amount of water to the above weight of remaining eleven raw materials respectively; decocting the first mixed solution and the second mixed solution twice respectively, wherein each time is one hour; obtaining a first filtrate and a second filtrate by filtering respectively; merging the first filtrate and the second filtrate, concentrating into an extract with a relative density in the range of 1.15-1.2 at 80-85 °C.

An administration method comprises steps of mixing the powders obtained by (S1) with the extract obtained by (S2), dissolving in water and administering orally.

### Second Embodiment

25 g of Radix rehmanniae, 13 g of Radix paeoniae rubra, 25 g of fructus gardeniae, 25 g of gypsum, 25 g of Sedum sarmentosum, 25 g of Solanum lyratum, 25 g of Oldenlandia diffusa, 25 g of Cornu bubali, 13 g of fructus forsythiae, 9 g of fried Rheum officinale, 6 g of liquorice, 0.9 g of antelope horn, 4.5 g of amber, and 3 g of Panax notoginseng are taken.

The preparation method and the administration method according to the second embodiment are the same as those according to the first embodiment.

### Third Embodiment

30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng are taken.

The preparation method and the administration method according to the third embodiment are the same as those according to the first embodiment.

### Fourth Embodiment

35 g of Radix rehmanniae, 17 g of Radix paeoniae rubra, 35 g of fructus gardeniae, 45 g of gypsum, 35 g of Sedum sarmentosum, 35 g of Solanum lyratum, 35 g of Oldenlandia diffusa, 45 g of Cornu bubali, 17 g of fructus forsythiae, 12 g of fried Rheum officinale, 12 g of liquorice, 1.8 g of antelope horn, 9 g of amber, and 6 g of Panax notoginseng are taken.

The preparation method and the administration method according to the fourth embodiment are the same as those according to the first embodiment.

### Fifth Embodiment

40 g of Radix rehmanniae, 20 g of Radix paeoniae rubra, 40 g of fructus gardeniae, 60 g of gypsum, 40 g of Sedum sarmentosum, 40 g of Solanum lyratum, 40 g of Oldenlandia diffusa, 40 g of Cornu bubali, 20 g of fructus forsythiae, 20 g of fried Rheum officinale, 15 g of liquorice, 2.4 g of antelope horn, 12 g of amber, and 8 g of Panax notoginseng are taken.

The preparation method and the administration method according to the fifth embodiment are the same as those according to the first embodiment.

### Sixth Embodiment: Animal Experiment on the Treatment of Immune Vasculitis with Traditional Chinese Medicine Composition Provided by the Present Invention

### Experimental Animals: Wistar Rats

Experimental medicine: the traditional Chinese medicine composition according to the third embodiment of the present invention the traditional Chinese medicine composition according to the third embodiment of the present invention

Experimental methods: The 4-week-old male Wistar rats are used as experimental animals. After one week of adaptive feeding, they are randomly divided into normal group, model group and administration group. The model group and the administration group are taken as the immune vasculitis model by injecting sodium laurate into the femoral artery. The administration group is given a certain dose of traditional Chinese medicine composition to intervene within 2 h after modeling, while the model group and the normal group are given an equal dose of drinking water. The dose given to rats is 6.1 times that given to the human body. 200 μl of the traditional Chinese medicine composition is intragastrically administered every day for a total of 14 days. The next day, the experimental rats in each group are treated and photographed, and the blood flow of lower limbs thereof is detected by ultra-high resolution spot scanning laser Doppler flow imager to evaluate the therapeutic effect of traditional Chinese medicine composition on immune vasculitis. At the same time, the heart, liver and kidney of these rats are taken and fixed with 4% paraformaldehyde, and the safety of cooling blood and circulating blood is evaluated by H&E (hematoxylin-eosin) staining.

Statistical methods: SPSS21.0 for windows statistical software is used for statistical analysis. Blood flow belongs to the measurement data, one-way classification ANOVA (analysis of variance) is used, and pairwise comparison is performed by t test (Student's t test), here, P<0.05 is used as the criterion of statistical difference.

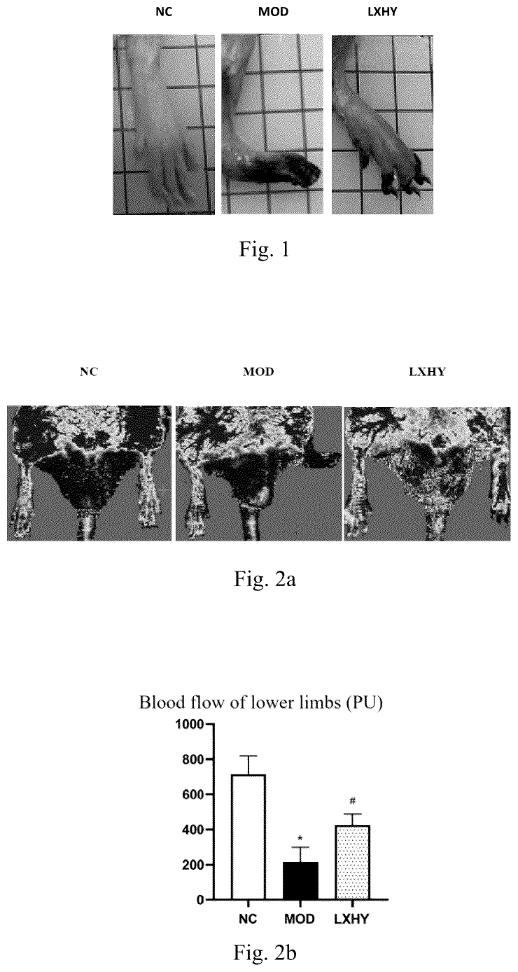

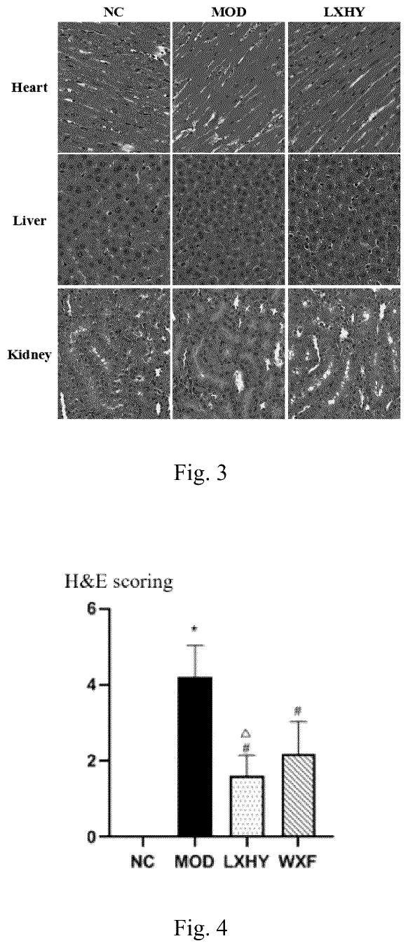

Experimental results: As shown in FIG. 1, compared with the normal group, the toes of the lower limbs of the rats in the model group all fall off, the foot stump is black, and the foot pad is red and swollen. In contrast, the traditional Chinese medicine composition significantly improves the degree of necrosis of the lower limbs of rats, only the foot tip is black, and the swelling degree of the foot pad is significantly reduced, which indicates that the traditional Chinese medicine composition is able to significantly improve the degree of necrosis of the lower limbs of rats. As shown in FIGS. 2a and 2b, it is found, by the ultra-high resolution point scanning laser Doppler flow imager, that the blood flow of lower limbs of rats in the model group is significantly lower than that in the normal group. Compared with the model group, the traditional Chinese medicine composition is able to significantly increase the blood flow of lower limbs of rats, which has the statistical difference (P<0.05). As shown in FIG. 3, no obvious abnormality is found in the heart, brain and kidney of the rats after treatment with the traditional Chinese medicine composition, which shows that the traditional Chinese medicine composition has no obvious toxic and side effects.

### Seventh Embodiment: Comparative Study of Traditional Chinese Medicine Composition and Literature Prescription

### Experimental Animals: Japanese Larger-Eared Juvenile Rabbits

Experimental medicine: the traditional Chinese medicine composition according to the third embodiment of the present invention

The literature prescription: 30 parts of Cornu bubali (decocted first), 15 g of Radix rehmanniae, 15 g of cortex moutan, 50 g of gypsum (decocted first), 20 g of lithospermum, 20 g of manyleaf paris rhizome, 30 g of Oldenlandia diffusa, 5 g of cooked rhubarb, 30 g of sculellaria barbata, 30 g of Radix actinidiae argutae, 20 g of sedum sarmentosum, and 15 g of liuyi powder (decocting in wrapped condition). The preparation method is accordance with the literature (Dongping Zhang, Jianchun Cao, “Treating Skin Vasculitis from Pathogenic Factor—Xi Jiuyi's Experience in Treating Skin Vasculitis” [J]., Chinese Journal of Integrative Surgery, 2004, 06:51-52.

Experimental methods: 4-week-old Japanese larger-eared juvenile rabbits are used as experimental animals. After one week of adaptive feeding, they are randomly divided into normal group, model group, traditional Chinese medicine composition group and literature group. The experimental rabbits in the model group, the traditional Chinese medicine composition group and the literature group are slowly injected with 25 ml/Kg of 10% bovine serum albumin by intravenous injection, and the normal group is given the equal dose of normal saline. Two weeks later, bovine serum albumin is injected again according to the above method to induce immune vasculitis. After the first modeling, on the next day, the traditional Chinese medicine composition and the literature prescription are respectively given for intervention, while the model group and the normal group are given the equal dose of drinking water. The dose of rabbits is 2.3 times that of human body. The intervention lasts for 14 days. On the next day, the coronary arteries are separated, and H&E staining and elastic fiber staining are performed to evaluate the therapeutic effect of the traditional Chinese medicine composition and the literature prescription on immune vasculitis.

Statistical methods: SPSS21.0 for windows statistical software is used for statistical analysis. Kruskal-Wallis H test, which provides the comparison of multiple samples in a completely random design, is used to compare the grade data, and Wilcoxon rank sum test is used for pairwise comparison, here, P<0.05 is used as the criterion of statistical difference.

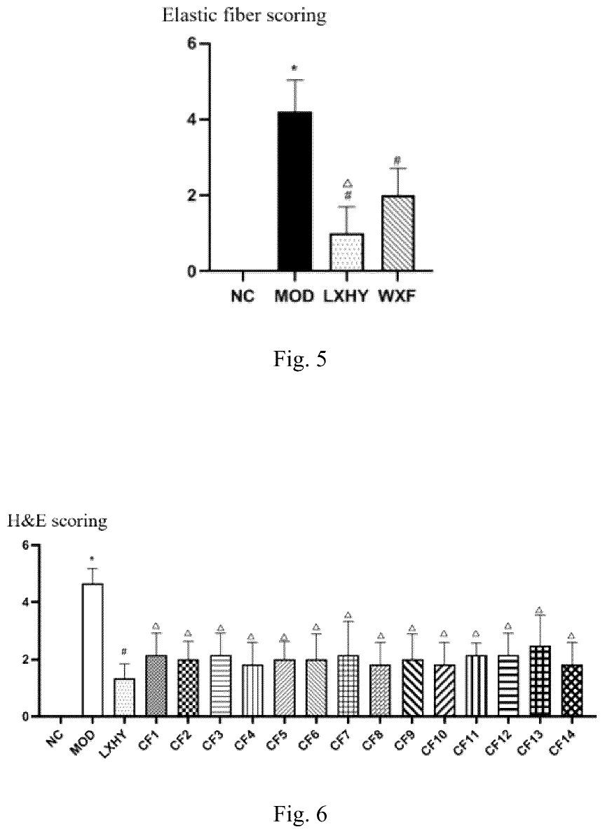

Experimental results: According to the degree of inflammatory cell infiltration, necrosis and detachment, and the arrangement and integrity of elastic fibers in arterial tissues, scoring is performed and scoring results are represented by “−, ±, +, ++, +++ and ++++”, here, “−” represents no obvious damage; “++++” represents extremely severe damage. As shown in FIGS. 4 and 5, compared with the model group, both the traditional Chinese medicine composition and the literature prescription significantly reduce the infiltration of inflammatory cells and significantly improve the lesions of elastic fibers, with statistical differences (P<0.05). However, the therapeutic effect of the traditional Chinese medicine composition is significantly better than that of the literature prescription, and there is statistical difference (P<0.05).

### Eighth Embodiment: Comparative Study on Traditional Chinese Medicine Composition and Separated Compound Prescription

### Experimental Animals: Balb/C Mice

Experimental medicine: traditional Chinese medicine composition according to the third embodiment of the present invention

The first separated compound prescription is composed of 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The second separated compound prescription is composed of: 30 g of Radix rehmanniae, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The third separated compound prescription is composed of 30 g of Radix rehmanniae, 15 g of Radix paeoniae rubra, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The fourth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The fifth separated compound prescription is composed of 30 g of Radix rehmanniae, 15 g of Radix paeoniae rubra, 15 g of fructus gardeniae, 30 g of gypsum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The sixth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The seventh separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

The eighth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 15 g of fructus forsythiae, 12 g of fried Rheum officinale, 9 g of liquorice, 1.2 g of antelope horn, 6 g of amber, and 4 g of Panax notoginseng.

The ninth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 12 g of fried Rheum officinale, 9 g of liquorice, 1.2 g of antelope horn, 6 g of amber, and 4 g of Panax notoginseng.

The tenth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 9 g of liquorice, 1.2 g of antelope horn, 6 g of amber, and 4 g of Panax notoginseng.

The eleventh separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 1.2 g of antelope horn, 6 g of amber, and 4 g of Panax notoginseng.

The twelfth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 6 g of amber, and 4 g of Panax notoginseng.

The thirteenth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, and 4 g of Panax notoginseng.

The fourteenth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, and 6 g of amber.

The above separated compound prescriptions are prepared by the preparation method according to the first embodiment of the present invention.

Experimental methods: Four-week-old female Balb/c mice are used as experimental animals. After one week of adaptive feeding, they are randomly divided into blank group, model group, traditional Chinese medicine composition group, and some separated compound prescription groups (first separated compound prescription, second separated compound prescription, third separated compound prescription, fourth separated compound prescription, fifth separated compound prescription, sixth separated compound prescription, seventh separated compound prescription, eighth separated compound prescription, ninth separated compound prescription, tenth separated compound prescription, eleventh separated compound prescription, twelfth separated compound prescription, thirteenth separated compound prescription and fourteenth separated compound prescription). Mice in the model group, the traditional Chinese medicine composition group, and each separated compound prescription are injected with 0.5 ml of 1 m/ml Lactobacillus casei cell wall components through the tail vein to create the immune vasculitis mouse model. The normal group is injected with the equal dose of PBS through the tail vein. On the next day after modeling, the traditional Chinese medicine composition and each separated compound prescription are administered respectively for intervention, while the model group and the normal group are administered the equal dose of drinking water. The dose administered to mice is 9.3 times that of human body. 200 μl of the above component is intragastrically administered every day. After 7 days of intervention, animals in each group are treated on the next day, and the H&E staining of tail vein is detected to evaluate the therapeutic effect of traditional Chinese medicine composition and each separated compound prescription on immune vasculitis.

Statistical methods: SPSS21.0 for windows statistical software is used for statistical analysis. Kruskal-Wallis H test, which provides the comparison of multiple samples in a completely random design, is used to compare the grade data, and Wilcoxon rank sum test is used for pairwise comparison, here, P<0.05 is used as the criterion of statistical difference.

Experimental results: According to the degree of inflammatory cell infiltration, necrosis and thrombosis in the tail vein, scoring is performed and scoring results are represented by “−, ±, +, ++, +++ and ++++”, here, “−” represents no obvious damage; “++++” represents extremely severe damage. As shown in FIG. 6, compared with the model group, both the traditional Chinese medicine composition and the separated compound prescription significantly reduce the infiltration of inflammatory cells, with statistical differences (P<0.05). However, the therapeutic effect of any separated compound prescription with less one ingredient is inferior to that of the traditional Chinese medicine composition, and there is statistical difference (P<0.05).

### Ninth Embodiment: Clinical Comparative Study of Traditional Chinese Medicine Compositions and Hormone

### 1. Clinical Data and Methods

### 1.1 Object of Study

All patients with immune vasculitis who are treated in the outpatient and inpatient departments of vascular of Shanghai Hospital of Integrated Traditional Chinese and Western Medicine.

### 1.2 Diagnostic Criteria

### 1.2.1 Western Diagnostic Criteria

The diagnosis is based on the 1990 ACR classification criteria and the 2012 Chapel-Hill Consensus Conference classification criteria.

### 1.2.2 Diagnostic Criteria of TCM (Traditional Chinese Medicine) Syndromes

The diagnosis refers to the Criteria for Diagnosis and Curative effect of Diseases of Traditional Chinese Medicine. The immune vasculitis belongs to blockade of damp-heat and static blood.

### 1.2.3 Inclusion Criteria

(1) It meets the diagnostic criteria of immune vasculitis; (2) It meets the blockade of damp-heat and static blood; (3) No age limit; (4) Both sexes; (5) Patients voluntarily participate in clinical trials and sign informed consent.

### 1.2.4 Exclusion Criteria

(1) Patients with hepatic and renal insufficiency, systemic infectious diseases, malignant tumors and mental diseases which may lead to severe complication; (2) Severe allergies; (3) Pregnant or lactating women; (4) Patients who are allergic to the experimental drug or its components.

### 1.2.5 Estimation of Sample Size

The estimation refers to the relevant formulas in the 6th edition of Medical Statistics. From the perspective of effectiveness, according to the statistical requirements, the optimal efficacy test and bilateral test are used, α=0.05, β=0.2, the expected average effective rate is 0.90, and the estimated number of cases is 126. Considering the shedding factors, 152 cases are determined in this experiment, and 76 cases are in the traditional Chinese medicine group and the Western medicine group, respectively.

### 1.2.6 Ethical Review

This study has been reviewed by the Ethics Committee of Shanghai Hospital of Integrated Traditional Chinese and Western Medicine, and obtained the ethics number ChiCTR2100052797.

### 1.3 Medical Treatment

Western medicine group: maintenance treatment with methylprednisolone tablets, 4-8 mg/day. Traditional Chinese medicine group: the traditional Chinese medicine composition provided by the present invention is given on the basis of 4-8 mg/day of methylprednisolone tablets.

The traditional Chinese medicine composition is prepared according to the third embodiment of the present invention. The antelope horn and Panax notoginseng in the traditional Chinese medicine composition provided by the present invention are purchased from Shanghai Wanshicheng State Medicine Products Co., LTD, China, the other raw materials are granules purchased from Jiangyin Tianjiang Pharmaceutical Co., Ltd., China. All of antelope horn, Panax notoginseng and the other raw materials are evenly mixed with boiled water and then taken orally twice.

The two groups are treated for 12 weeks and followed up for half a year.

### 1.4 Observational Indexes

Birmingham systemic vasculitis activity Score (BVAS) is performed on patients in each group before and after treatment, and the patients are followed up to observe the occurrence of Cushing's syndrome such as central obesity, full moon face and increased hair.

The curative effect evaluation is evaluated according to the curative effect index. The curative effect index=(total scores before treatment-total scores after treatment)/total scores before treatment. (1) Obvious effect: the symptoms are significantly improved, and the syndrome score is reduced by ≥70% compared with that before treatment; (2) Effective: the symptoms are improved, and the syndrome score is reduced by ≥30% and <70% compared with that before treatment; (3) Ineffective: the symptoms are not improved, and the syndrome score is decreased by <30% compared with that before treatment; (4) Aggravation: the symptoms are aggravated, and the syndrome score is reduced by <0%.

Statistical methods: SPSS21.0 for windows statistical software is used for statistical analysis. The adoption rate and composition ratio of counting data are expressed by x² test, here, P<0.05 is used as the criterion of statistical difference.

### 2. Results

As shown in Table 1, a total of 152 cases of immune vasculitis are collected, including 76 cases in the traditional Chinese medicine group and 76 cases in the western medicine group. The total effective rate of the traditional Chinese medicine group is 97.34%, which is significantly higher than that of the Western medicine group, with statistical difference (P<0.05). And through follow-up, it is found that after taking methylprednisone tablets in the western medicine group, there are 13 patients with different degrees of Cushing's syndrome, while no cases of Cushing's syndrome are found in the traditional Chinese medicine group. It is able to be seen that the combination of traditional Chinese medicine composition and western medicine is effective in the treatment of immune vasculitis, and the traditional Chinese medicine composition is also able to significantly prevent the Cushing's syndrome caused by hormone (methylprednisolone tablets) treatment.

TABLE 1
Comparison of clinical efficacy between two groups (cases)

| | | Cases | Cases | | Total | The number |
|---|---|---|---|---|---|---|
| | | with | with | Cases with | effective | of cases with |
| | Total | obvious | effective | ineffective | ratio | Cushing's |
| Group | cases | effect | result | result | (%) | syndrome |
| Traditional | 76 | 59 | 15 | 2 | 97.34 | — |
| Chinese | | | | | | |
| Medicine | | | | | | |
| Group | | | | | | |
| Western | 76 | 34 | 28 | 14 | 81.58 | 13 |
| Medicine | | | | | | |
| Group | | | | | | |

### Tenth Embodiment: Clinical Study on the Treatment of Various Immune Vasculitic Diseases with Traditional Chinese Medicine Composition

Experimental methods: The inventor collects 95 cases of immune vasculitis, including 30 cases of nodular vasculitis, 10 cases of Takayasu arteritis, 10 cases of thromboangiitis obliterans, 15 cases of Rheumatoid vasculitis, 10 cases of systemic lupus erythematosus vasculitis, 10 cases of scleroderma vasculitis, and 10 cases of ACNA associated vasculitis. They are treated with the traditional Chinese medicine composition (according to the ninth embodiment of the present invention) for 14 days, and the Birmingham Systemic Vasculitis Activity Score (BVAS) is completed before and after treatment to evaluate the therapeutic effect of the traditional Chinese medicine composition. As shown in Table 2, the traditional Chinese medicine composition significantly improves the inflamed and pain symptoms in various patients.

TABLE 2
Clinical observation on the therapeutic effect of traditional Chinese
medicine composition on various immune vasculitis diseases

| | | Cases | Cases | Cases | Total |
|---|---|---|---|---|---|
| | | with | with | with | effective |
| | | obvious | effective | ineffective | ratio |
| Group | Cases | effect | result | result | (%) |
| Nodular | 30 | 26 | 2 | 2 | 93.33 |
| vasculitis | | | | | |
| Takayasu | 12 | 8 | 3 | 1 | 91.67 |
| arteritis | | | | | |
| Thromboangiitis | 17 | 14 | 2 | 1 | 94.12 |
| obliterans | | | | | |
| Rheumatoid | 15 | 12 | 1 | 2 | 86.67 |
| vasculitis | | | | | |
| Systemic | 11 | 7 | 3 | 1 | 90.91 |
| lupus | | | | | |
| erythematosus | | | | | |
| vasculitis | | | | | |
| Scleroderma | 13 | 8 | 4 | 1 | 92.31 |
| vasculitis | | | | | |
| ACNA | 9 | 5 | 3 | 1 | 88.89 |
| associated | | | | | |
| vasculitis | | | | | |

Many aspects of the present invention are described above. However, it should be understood that, without deviating from the spirit of the present invention, equivalent alterations and modifications may be made by those skilled in the art, which also fall within the protection scope of the claims of this application. The fourth separated compound prescription is composed of 30 g of Radix rehmanniae, 15 g of Radix paeoniae rubra, 15 g of fructus gardeniae, 30 g of gypsum, 30 g of Sedum sarmentosum, 30 g of Solanum lyratum, 30 g of Oldenlandia diffusa, 30 g of Cornu bubali, 15 g of fructus forsythiae, 12 g of fried Rheum officinale, 9 g of liquorice, 1.2 g of antelope horn, 6 g of amber, and 4 g of Panax notoginseng.

1. A traditional Chinese medicine composition for treating immune vasculitis, which comprises following raw material components by weight:
15-40 parts of Radix rehmanniae, 10-20 parts of Radix paeoniae rubra, 10-40 parts of fructus gardeniae, 15-60 parts of gypsum, 15-40 parts of Sedum sarmentosum, 15-40 parts of Solanum lyratum, 15-40 parts of Oldenlandia diffusa, 15-40 parts of Cornu bubali, 10-20 parts of fructus forsythiae, 6-20 parts of fried Rheum officinale, 3-15 parts of liquorice, 0.6-2.4 parts of antelope horn, 3-12 parts of amber, and 2-8 parts of Panax notoginseng.

2. The traditional Chinese medicine composition according to claim 1, wherein the traditional Chinese medicine composition comprises following raw material components by weight: 30 parts of Radix rehmanniae, 15 parts of Radix paeoniae rubra, 15 parts of fructus gardeniae, 30 parts of gypsum, 30 parts of Sedum sarmentosum, 30 parts of Solanum lyratum, 30 parts of Oldenlandia diffusa, 30 parts of Cornu bubali, 15 parts of fructus forsythiae, 12 parts of fried Rheum officinale, 9 parts of liquorice, 1.2 parts of antelope horn, 6 parts of amber, and 4 parts of Panax notoginseng.

3. The traditional Chinese medicine composition according to claim 1, wherein the traditional Chinese medicine composition comprises following raw material components by weight: 20 parts of Radix rehmanniae, 10 parts of Radix paeoniae rubra, 20 parts of fructus gardeniae, 20 parts of gypsum, 20 parts of Sedum sarmentosum, 20 parts of Solanum lyratum, 20 parts of Oldenlandia diffusa, 20 parts of Cornu bubali, 10 parts of fructus forsythiae, 6 parts of fried Rheum officinale, 3 parts of liquorice, 0.6 parts of antelope horn, 3 parts of amber, and 2 parts of Panax notoginseng.

4. The traditional Chinese medicine composition according to claim 1, wherein the traditional Chinese medicine composition comprises following raw material components by weight: 25 parts of Radix rehmanniae, 13 parts of Radix paeoniae rubra, 25 parts of fructus gardeniae, 25 parts of gypsum, 25 parts of Sedum sarmentosum, 25 parts of Solanum lyratum, 25 parts of Oldenlandia diffusa, 25 parts of Cornu bubali, 13 parts of fructus forsythiae, 9 parts of fried Rheum officinale, 6 parts of liquorice, 0.9 parts of antelope horn, 4.5 parts of amber, and 3 parts of Panax notoginseng.

5. The traditional Chinese medicine composition according to claim 1, wherein the traditional Chinese medicine composition comprises following raw material components by weight: 35 parts of Radix rehmanniae, 17 parts of Radix paeoniae rubra, 35 parts of fructus gardeniae, 45 parts of gypsum, 35 parts of Sedum sarmentosum, 35 parts of Solanum lyratum, 35 parts of Oldenlandia diffusa, 45 parts of Cornu bubali, 17 parts of fructus forsythiae, 12 parts of fried Rheum officinale, 12 parts of liquorice, 1.8 parts of antelope horn, 9 parts of amber, and 6 parts of Panax notoginseng.

6. The traditional Chinese medicine composition according to claim 1, wherein the traditional Chinese medicine composition comprises following raw material components by weight: 40 parts of Radix rehmanniae, 20 parts of Radix paeoniae rubra, 40 parts of fructus gardeniae, 60 parts of gypsum, 40 parts of Sedum sarmentosum, 40 parts of Solanum lyratum, 40 parts of Oldenlandia diffusa, 40 parts of Cornu bubali, 20 parts of fructus forsythiae, 20 parts of fried Rheum officinale, 15 parts of liquorice, 2.4 parts of antelope horn, 12 parts of amber, and 8 parts of Panax notoginseng.

7. A Chinese herbal medicine compound preparation for treating immune vasculitis, which is a decoction, a granule, a pulvis, a capsule, a tablet, or an oral liquid with the traditional Chinese medicine composition according to claim 1 as an active component.

8. The Chinese herbal medicine compound preparation according to claim 7, wherein the Chinese herbal medicine compound preparation is the decoction.

9. A method for treating immune vasculitis disease comprising a step of applying the traditional Chinese medicine composition according to claim 1, wherein the immune vasculitis disease is nodular vasculitis, aorto-arteritis, thromboangiitis obliterans, Rheumatoid vasculitis, systemic lupus erythematosus vasculitis, scleroderma vasculitis, or ACNA (antineutrophil cytoplasmic antibodies)-related vasculitis.

10. (canceled)
